(19) United States
(12) Patent Application Publication
(10) Pub. No.: US 2022/0000564 A1
(43) Pub. Date: Jan. 6, 2022

(54) METHODS AND SYSTEMS FOR SURGICAL NAVIGATION AND DEVICES FOR SURGERY

(71) Applicant: INTELLIJOINT SURGICAL INC., KlTCHENER (CA)

(72) Inventors: SAMANTHA MCCABE, KITCHENER (CA); RICHARD TYLER FANSON, STONEY CREEK (CA); ANDRE NOVOMIR HLADIO, WATERLOO (CA)

(21) Appl. No.: 17/291,526

(22) PCT Filed: Nov. 6, 2019
(86) PCT No.: PCT/CA2019/051578

§ 371 Date: May 5, 2021

Related U.S. Application Data
(60) Provisional application No. 62/756,786, filed on Nov. 7, 2018.

(51) Int. Cl. A61B 34/20; A61B 34/00; A61B 17/15; A61B 17/17

(52) U.S. Cl. CPC A61B 34/20; A61B 34/25; A61B 17/155; A61B 2017/00128; A61B 17/1746; A61B 17/175; A61B 17/1757; A61B 17/157

(57) ABSTRACT

Provided are methods and systems for surgical navigation including audible cues to position a tracked surgical device relative to patient anatomy. Also provided are surgical devices, such as a cutting guide, having independent adjustability to meet target parameters. A separable paddle guide mountable to the cutting guide may be provided to position the cutting guide in a first position. A kit may be provided such as a cutting guide, paddle guide and optionally pins and a tracking element.

### CROSS-REFERENCE

This application is a National Stage of International Patent Application No. PCT/CA2019/051578, filed Nov. 6, 2019, the entire content of which is incorporated herein by reference. International Patent Application No. PCT/CA2019/051578 claims the benefit of and/or priority to U.S. Provisional Application No. 62/756,786 filed Nov. 7, 2018, the content of which is incorporated herein by reference in its entirety.

### FIELD

The present disclosure relates to, in one aspect, computing methods and systems, namely methods and systems for surgical navigation such as to guide a procedure, assisting to place a surgical tool such as a cutting guide or other object, and, in another, to devices for surgery, namely a cutting guide or jig.

### BACKGROUND

Many surgeons like to focus their view on the surgical site, whereas many surgical navigation systems have display screens with spatial information that are located away from the patient in the operating room.

Mounting a cutting guide or other device to meet target parameters may be tricky or slow. It may be desired to provide a device that is amenable to adjustment to assist with mounting while meeting the target parameters. The structure of the device itself or attachments therefor may assist with mounting and help to determine an order or process of mounting to meet the target parameters.

### SUMMARY

Provided are methods and systems for surgical navigation including audible cues to position a tracked surgical device relative to patient anatomy. Also provided are surgical devices, such as a cutting guide, having independent adjustability to meet target parameters.

In one aspect, to permit a surgeon to visually focus on the surgical site while receiving information from a surgical navigation system that is tracking one or more objects at the site, there is provided a surgical navigation system that uses audio feedback to communicate spatial information to a surgeon. Surgeons can focus their view on the surgical site, while leveraging the benefits of the accuracy of the surgical navigation system.

In another aspect, the device to be spatially positioned with respect to the patient (e.g. a cutting guide) may provide means for independent adjustment of each measured parameter. A separable paddle guide mountable to the cutting guide may be provided to position the cutting guide in a first position. A kit may be provided such as a cutting guide, paddle guide and optionally pins and a tracking element.

These and other aspects will be understood to a person of skill in the art and such a person also understand that the aspects may be summarized or described separately for convenience, they may be combined and/or used together.

### BRIEF DESCRIPTION OF THE DRAWINGS

One or more embodiments are shown and/or described with references to drawings in which:

FIG. 1A is a sketch of a representative operating room showing a draped patient and a computing system of a (knee) navigation system in accordance with an example;

FIG. 1B shows an enlarged cutting guide according to the example of FIG. 1A; and

FIG. 2 is a block diagram of an example computing device such as for a computing system of FIG. 1A;

FIGS. 3 and 4 show images of a first embodiment of a cutting guide;

FIG. 5 shows an image of a first embodiment of paddle guide;

FIG. 6 shows the paddle guide of FIG. 5 mounted to the cutting guide of FIG. 3 in an assembly;

FIG. 7 a tracking element mounted to the assembly of FIG. 6;

FIG. 8A is a lateral schematic view of a knee where the dotted lines show flexion and extension movement and the solid line shows resection depth along a femur;

FIG. 8B is a frontal schematic view of a knee where the dotted lines show varus/valgus angle and the solid line shows resection depth along a femur;

FIGS. 9A to 9F show a model receiving a procedure using the cutting guide, paddle guide and tracking element shown in FIG. 7 according to an embodiment. It is noted that not all of these three components are shown in each of these figures;

FIG. 10 shows an image of a second embodiment of a cutting guide;

FIGS. 11A and 11B are CAD images of an extension member of the cutting guide of FIG. 3;

FIG. 12 is a CAD image of a body of the cutting guide of FIG. 3;

FIG. 13 is a cross-sectional view, orthogonal to a longitudinal axis showing the assembly of the body and extension member viewed of the cutting guide of FIG. 3;

FIGS. 14A to 14C are CAD images of the cutting guide of FIG. 3 in an assembly with a tracking element;

FIGS. 15A to 15C are CAD images of the cutting guide of FIG. 3 with the first embodiment of the paddle guide of FIG. 5 in an unassembled and assembled state;

FIG. 15D is a CAD image of the assembled state of FIG. 15B in a further assembly with a tracking element and shown mounted on a femur;

FIG. 16A is a CAD image of a second embodiment of a paddle guide;

FIG. 16B is a CAD image of the second embodiment of the paddle guide in an assembly with the first embodiment of the cutting guide;

FIG. 16C is a CAD image of the assembly of FIG. 16B further assembly with a tracking element and shown mounted on a femur;

FIG. 17A is a CAD image of a third embodiment of a paddle guide; and

FIG. 17B is a CAD image of the third embodiment of the paddle guide in an assembly with the first embodiment of the cutting guide and in a further assembly with a tracking element and shown mounted on a tibia.

### DETAILED DESCRIPTION

In total knee arthroplasty (TKA), a distal femoral cut involves three target parameters: varus/valgus angle, flexion/extension angle, and resection depth. Each parameter should be made (determined and set) accurately and precisely so that the surgery restores the patient's biomechanics or otherwise determines the biomechanics in a desired way.

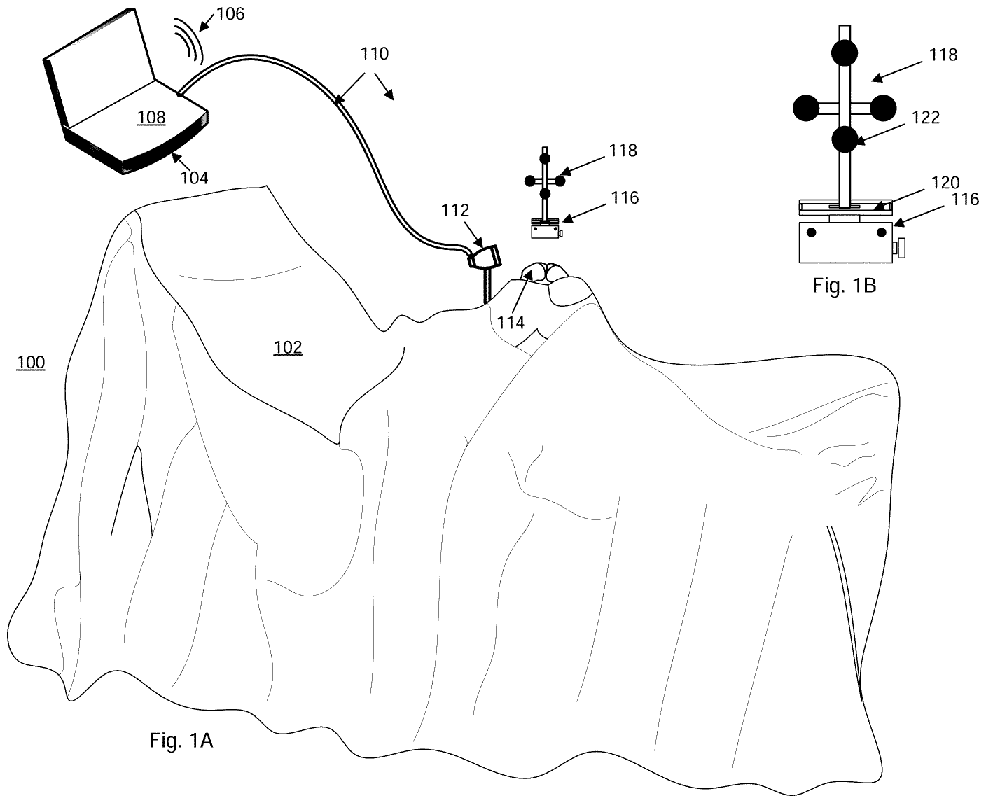

A knee navigation system can be used for the surgery, involving a reference element attached to the patient's femur. The patient's femur may be registered to the navigation system relative to the reference element. FIG. 1A is a representative operating room 100 showing a draped patient 102 and a computing system 104 of a (knee) navigation system 110 which system 110 further comprises a reference element (e.g. an optical device 112) attached to a femur 114. As a part of the procedure, guidance is provided to position a surgical tool, namely a cutting guide 116, in a target position defined by target parameters input or otherwise received by the computing system 102. The cutting guide is coupled to a tracking element 118. FIG. 1B shows an enlarged cutting guide 118.

The cutting guide 118 may provide a slot 120 to receive a cutting blade of an oscillating saw (resection tool) (not shown here); the relative position and orientation (pose) of the slot relative to the femur defines the varus/valgus angle, the flexion/extension angle and the resection depth. Based on clinical norms, implant design, and pre-operative planning, there may be target parameters for varus/valgus angle, flexion/extension angle, and resection depth. While a slot is shown, the slot provides a planar blade guide surface to receive and guide a cutting blade such that a slot per se (a closed planar aperture) need not be formed and as a planar blade guide surface may be sufficient.

The tracking element 118 may be coupled to the cutting guide in a known relationship such that the pose of the tracking element 118 relates to the pose of the slot 120. The tracking element may be coupled temporarily while placing the cutting guide on the femur and removed once placed.

The navigation system 110 may measure the relative pose between reference element 112 and the tracking element 118, and may compute the relative pose of the slot and the femur based on registration information.

The navigation system 110 may receive input comprising the target parameters. Input may be received via an input device such as a keyboard, touch screen, microphone, optical device reading a stored data in a 2D or 3D barcode (e.g. a quick reference code), a communication from another computing device for example a device providing patient and related pre-operative and other information, a removable storage device (e.g. a thumb drive), or any other input.

The navigation system 110 may compute respective varus/valgus angle, the flexion/extension angle and the resection depth measurements using the relative pose of the slot and the femur and compute one or more difference measures between these measurements and the target parameters received.

Using the difference measures, audio signals 106 are output by the navigation system 110 such via a speaker device 108 of computing device 104 (or a speaker otherwise coupled thereto) to indicate how close the current measured parameters (e.g. flexion/extension angle) are to target parameters. As a surgeon adjusts the cutting guide into place, the audio output may guide him or her to achieve the target parameters.

Audio signals 106 may comprise a series of beeps. The audio signals may vary with the magnitude of the differences, for example in a similar manner to how a Geiger counter varies its audio signal with a relative strength (e.g. an ionizing event count) of the ionizing radiation signal (e.g. ionizing incidents) it is measuring. The closer the cutting guide is moved to a desired position (where the difference between measured values and target parameters is minimized), the louder and faster (more frequently) the audio signal may beep. As the cutting guide is moved away from the target, the audio signal may beep less frequently and less loudly. The audio signal may be configured to in an opposite manner, beep less frequently and less loudly and once the target is meet, a bell sound may be output. Other audio signals may be used which may vary pitch, volume, beat and/or rhythm, and/or other attributes. A threshold may be determined viz. the difference such that the measured values need not be identical with the target parameters. A bell or other sound may be made when the measured value is within the threshold difference.

Measurements cannot be made when sensors (e.g. 122) of the tracking element are not in a field of view of a tracking device such as an optical device (camera). Typical tracking elements comprise passive sensors (e.g. reflective trackers or markers) which reflect light in an IR spectrum or otherwise to an optical device. Active sensors may be used that emit a source light to the optical device. The position of the passive or active sensors (i.e. the light therefrom captured in the image) in an image by the optical device determines the pose of the tracking element and the object to which it is coupled. When these sensors are not visible in the image, measurement cannot be undertaken, at least in the absence of other sensors. An audible signal, e.g. a distinct signal, may be output when the tracking element (e.g. one or more sensors thereon) is not in view.

Adjustments of the cutting guide coupled to the tracking element may be made in a manner to allow the navigational system to perform the measurements and output the sound before the cutting guide is moved to a next position that is not as optimal.

Audio signals (e.g. providing audio cues) may be output from a speaker of the navigational system or to headphones coupled thereto, etc. Audio signals may be selectively turned on an off and permit a (baseline) volume adjustment. In some examples, such an adjustment or disabling feature may not be allowed. For example, certain audio controls may be disabled and/or forced on while the audio signal function is in use.

Audio cues may only play when the cutting guide is in a ball park (e.g. position and angle) of the target. “Ball park” means within a respective threshold measure of one or more target parameters, for example, within 5 cm of a target resection and within 45 degrees of the respective angles. It may be necessary to meet more than one threshold measure before audio cues are provided (initiated). The positioning within the threshold measures can be determined via registration information associated with the reference element and a relative pose of the reference element and the tracking element.

Once the cutting guide is in place. The tracking element may be removed.

It may be preferred to place a guide in a staged or phased manner, whereby the guide is initially positioned and its initial position adjusted to satisfy each target parameter in turn. For example, it may be preferred to set the flexion/extension angle, hold this position fixed, set the varus/valgus angle, hold the position fixed to maintain both parameters set and then position the guide to set the resection depth before finally securing the position of the cutting guide such as by fixing the guide to the femur or other bone using one or more pins or screws.

While the above description relates to mounting a cutting guide to a femur, the pose of other objects relative to a patient's anatomy may be guided using an audio output signal as described. Examples in other surgical applications may include:
i. THA—acetabular cup placement or broach placement and target parameters and measured parameters may be chosen from
   a. Leg length;
   b. Offset;
   c. Acetabular inclination;
   d. Acetabular anteversion;
   e. Acetabular Reaming Depth; and
   f. Broach depth.
ii. Spine pedicle screw placement and the target parameters and measured parameters are for aligning a trajectory of a tool with a pedicle of the patient
iii. Knee—Proximal tibia resection
iv. Etc.

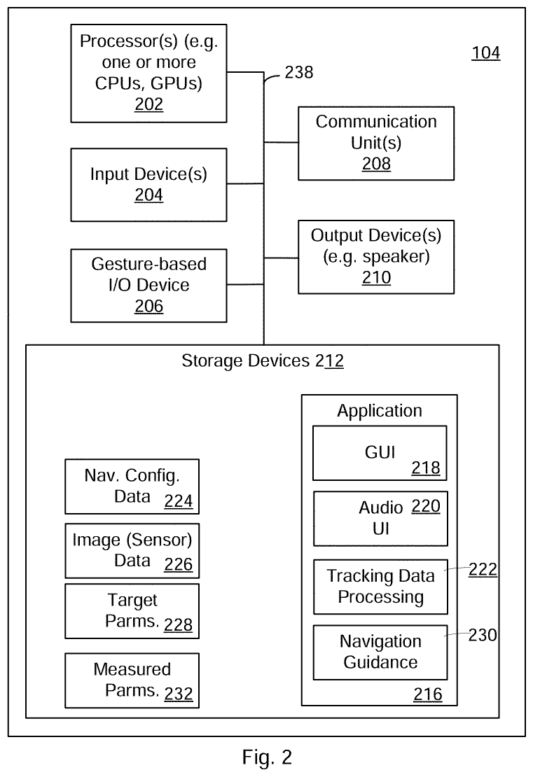

FIG. 2 is a block diagram of an example computing device 200, in accordance with one or more aspects of the present disclosure, such as a laptop, workstation, tablet, etc. Computing device 104 comprises one or more processors 202, one or more input devices 204, a gesture-based I/O device 206, one or more communication units 208 and one or more output devices 210. Computing device 104 also includes one or more storage devices 212 storing one or more modules and/or data. Modules may a surgical navigation application (e.g. application 216) having components or modules for a graphical user interface (GUI 218), an audio user interface 220, tracking data processing 222 and navigation guidance 230. Data may include navigation system configuration data 224, one or more images for processing (e.g. image (sensor) data 226), target parameters 228 and measured parameters (e.g. 232).

Application 216 provides the functionality (e.g. via tracking data processing component 222) to acquire one or more images (e.g. 226) from the optical sensor (e.g. camera 112), such as a video and process the images to determine pose information for target element 118 and hence slot 120. Navigation guidance component 230 may determine measurement parameters 230 for comparison to target parameters 228 to provide navigational guidance such as audio signals via audio UI 220 and a speaker device of output devices 210) and/or graphical signals via GUI 218. Tracking data processing may use navigational configuration data such as for relationships between the sensors of tracking element 118 and its base (i.e. the relationship between tracking element 118 and slot 120. It may also perform a registration. Application 216 may be programmed with workflow to perform a process and guide a user through a sequence of steps to perform a procedure or any part thereof. The GUI may be provided via gesture-based I/O device 206 or another display device (e.g. one of output devices 210).

Storage device(s) 212 may store additional modules such as an operating system and other modules (not shown) including communication modules; graphics processing modules (e.g. for a GPU of processors 202); pre-operative planning modules, etc. Storage devices may be referenced as storage units herein.

Communication channels 238 may couple any of the components and modules shown in FIG. 2 inter-component communications, whether communicatively, physically and/or operatively. In some examples, communication channels 238 may include a system bus, a network connection, an inter-process communication data structure, or any other method for communicating data.

The one or more processors 202 may implement functionality and/or execute instructions within computing device 104. For example, processors 202 may be configured to receive instructions and/or data from storage devices 212 to execute the functionality of the modules shown in FIG. 2, among others (e.g. operating system, applications, etc.) Computing device 104 may store data/information to storage devices 212. Some of the functionality is described further herein below. It is understood that operations may not fall exactly within the components of application 816 as shown such that one may assist with the functionality of another and/or may receive assistance from components such as an operating system or communication module or other component/module not shown.

Computer program code for carrying out operations may be written in any combination of one or more programming languages, e.g., an object oriented programming language such as Java, Smalltalk, C++ or the like, or a conventional procedural programming language, such as the “C” programming language or similar programming languages.

Computing device 104 may generate output for display on a screen of gesture-based I/O device 206 or in some examples, for display by a projector, monitor or other display device. It will be understood that gesture-based I/O device 206 may be configured using a variety of technologies (e.g. in relation to input capabilities: resistive touchscreen, a surface acoustic wave touchscreen, a capacitive touchscreen, a projective capacitance touchscreen, a pressure-sensitive screen, an acoustic pulse recognition touchscreen, or another presence-sensitive screen technology; and in relation to output capabilities: a liquid crystal display (LCD), light emitting diode (LED) display, organic light-emitting diode (OLED) display, dot matrix display, e-ink, or similar monochrome or color display).

In the examples described herein, gesture-based I/O device 206 includes a touchscreen device capable of receiving as input tactile interaction or gestures from a user interacting with the touchscreen. Such gestures may include tap gestures, dragging or swiping gestures, flicking gestures, pausing gestures (e.g. where a user touches a same location of the screen for at least a threshold period of time) where the user touches or points to one or more locations of gesture-based I/O device 206. Gesture-based I/O device 206 and may also include non-tap gestures. Gesture-based I/O device 206 may output or display information, such as graphical user interface, to a user. The gesture-based I/O device 206 may present various applications, functions and capabilities of the computing device 104 including, for example, application 216 to present GUI 218.

Although the present disclosure illustrates and discusses a gesture-based I/O device 206 primarily in the form of a display screen device with I/O capabilities (e.g. touchscreen), other examples of gesture-based I/O devices may be utilized which may detect movement and which may not comprise a screen per se. In such a case, computing device 104 includes a display screen or is coupled to a display apparatus to present GUI 218. Computing device 104 may receive gesture-based input from a track pad/touch pad, one or more cameras, or another presence or gesture sensitive input device, where presence means presence aspects of a user including for example motion of all or part of the user.

One or more communication units 208 may communicate with external devices (not shown) for example to receive config. data, target parameters, or other data such as from coupled optical sensor 112 or application functionality, to share data with another computing device, printing device or display device (all not shown) via one or more communication networks (not shown) by transmitting and/or receiving network signals on the one or more networks. The communication units may include various antennae and/or network interface cards, chips (e.g. Global Positioning Satellite (GPS)), etc. for wireless and/or wired communications.

Input devices 204 and output devices 210 may include any of one or more buttons, switches, pointing devices, cameras, a keyboard, a microphone, one or more sensors (e.g. biometric, etc.), a speaker, a bell, one or more lights, a haptic (vibrating) device, etc. One or more of same may be coupled via a universal serial bus (USB) or other communication channel (e.g. 238).

The one or more storage devices 212 may take different forms and/or configurations, for example, as short-term memory or long-term memory. Storage devices 212 may be configured for short-term storage of information as volatile memory, which does not retain stored contents when power is removed. Volatile memory examples include random access memory (RAM), dynamic random access memory (DRAM), static random access memory (SRAM), etc. Storage devices 212, in some examples, also include one or more computer-readable storage media, for example, to store larger amounts of information than volatile memory and/or to store such information for long term, retaining information when power is removed. Non-volatile memory examples include magnetic hard discs, optical discs, floppy discs, flash memories, or forms of electrically programmable memory (EPROM) or electrically erasable and programmable (EEPROM) memory.

Thus, there is provided a computer method for a guided procedure on a patient. The method comprises: receiving at least one target parameter; determining at least one measured parameter in real time using data received from a navigation system; generating an audio signal indicative of a proximity of the measured parameter to the target parameter; and providing the audio signal to a speaker device.

The audio signal may indicate a proximity of the measured parameter with the target parameter in various manners, including any of: a beeping tone wherein a frequency is one of proportional and inversely proportional to a magnitude of difference between the measured parameter and the target parameter; a different signal indicating if the difference is positive or negative where the difference is positive when the measured parameter is greater than the target parameter and negative when less than the target parameter; a distinct audio signal when the target parameter =measured parameter (within a threshold); a distinct audio signal when the measured parameter is grossly out of range of the target parameter such that the difference is >> an out of range threshold; an a distinct audio signal indicating a “Not Tracking” state of the navigation system, whereby the navigation system is unable to produce the data to determine the measured parameter.

There may be two or more target parameters associated with two or more measured parameters. The audio signal may be generated based on only one of the two or more target parameters associated with the two or more measured parameters. The method may include receiving user input to switch between one of the two or more target parameters and another one of the two or more target parameters and providing the audio signal accordingly.

The method may include providing a user interface to receive input to configure the audio signal. An option to configure the audio signal may comprise an option to turn on/off audio output and/or adjust volume.

Both the target parameter and the measured parameter may be provided for display to a display screen and the display may comprise one or more of graphical or numerical representation.

When two or more target parameters are present, one of the two or more target parameters may be displayed and graphically emphasized (e.g. when the one is selected as the parameter associated with the audio signal. The one of the two or more target parameters may be emphasized by: an increased brightness on the display screen; a coloured or thicker border; and a graphical element associated with of the two or more target parameters that changes its visual appearance synchronously with the audio signal (e.g. a flashing border that flashes with each beep).

The guided procedure may be a Total Knee Replacement. The at least one target and at least one measured parameter may be chosen from: varus/valgus angle; flexion/extension angle; resection depth; and overall leg alignment.

The guided procedure may be a Total Hip Arthroplasty. The at least one target parameter and at least one measured parameter may be chosen from: leg length; offset; acetabular inclination; acetabular anteversion; acetabular reaming depth; and broach depth.

The procedure may comprise a pedicle screw placement. The at least one target parameters are useful to align a trajectory of a tool with a pedicle of the patient.

There is provided a computing device configured to perform the method(s) or any of the method features as described, whether individually or in combination unless the context requires otherwise.

There is provided a computer program product comprising a non-transient storage unit (e.g. a device such as a memory, disc/disk, etc.) storing instructions which when executed by a processing unit of a computing device configure the computing device to perform the method(s) or any of the method features as described, whether individually or in combination unless the context requires otherwise.

### Cutting Guide

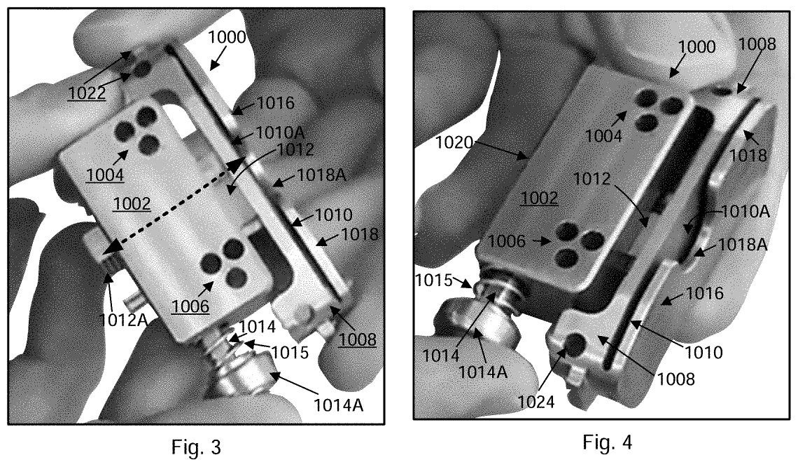

A cutting guide to be spatially positioned may be provided with structure to enable independent adjustment of each measured parameter. For example, if the device is a knee cutting guide, a first pin may be used to effect flexion/extension angle; a second pin may be used to effect a varus/valgus angle (independent of the flexion/extension angle); and a translational mechanism may be used to effect resection depth (independent of both preceding measured parameters). Reference may be had to FIGS. 3 and 4 showing a cutting guide 1000. Cutting guide 1000 has a body 1002, with a longitudinal axis as indicated by the dotted line. Extending through opposite surfaces of the guide 1000 are formed a first cluster of pin holes 1004 (e.g. on a left side) and opposite and parallel thereto, across the longitudinal axis are formed a second cluster of pin holes 1006 (e.g. on a right side). The pin holes are configured to receive a pin to mount the cutting guide, as further described, to a bone such as an anterior femur or anterior tibia for respective knee procedures. It is understood that two pins are received in two pin holes, one from each cluster.

The pin holes in each cluster are relatively spaced with one another in two offset columns to provide different but relatively close spacing from a top end surface 1016 of the cutting guide. In a respective cluster, a first pin hole may be spaced an initial distance X mm from the top surface, a second spaced (X+Y) mm and a third spaced (X+2Y) mm from the top surface, etc. to give measured adjustment. The Y value may be 1 for example, to allow 1 mm adjustment. Though a cluster is shown, only one pin hole may be necessary on each of the left side and right side (two pin holes in total).

Adjacent the top end surface and transverse to the longitudinal axis there is formed a planar slot 1010 through the cutting guide device. A surface defining the slot also provide a planar blade guide surface 1010A. In the present example, body 1002 carries a head 1008 in which the slot 1010 is formed. Slot 1010 may receive a cutting blade of an oscillating saw (not shown). The respective positions of the pin holes 1004 and 1006 are spaced from and parallel to slot 1010. Parallel positioning may be preferred but is not necessary

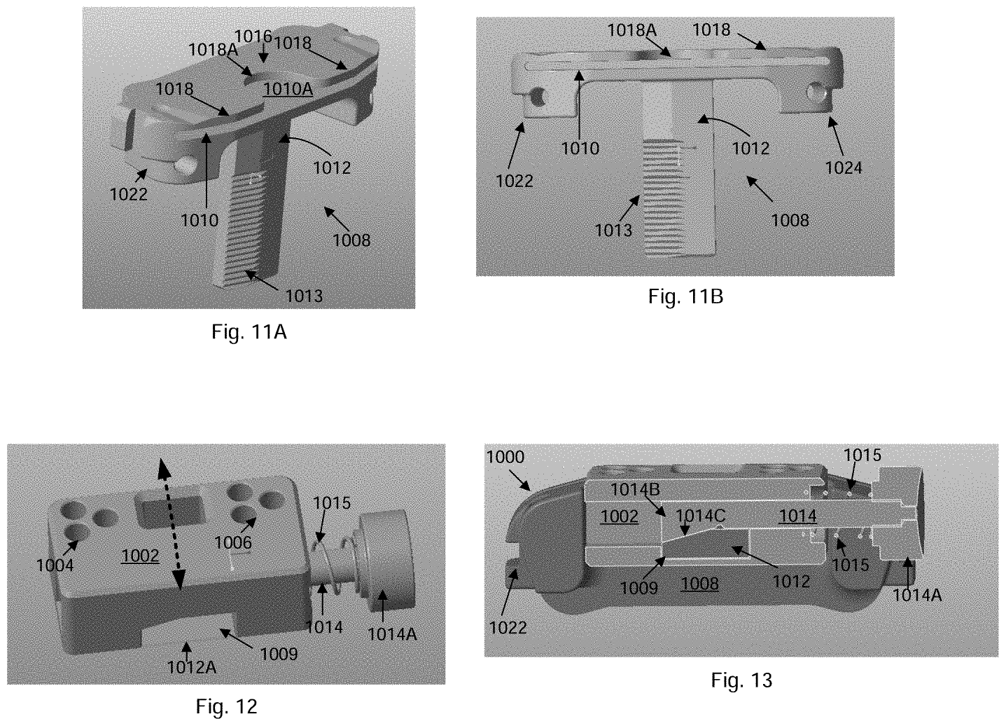

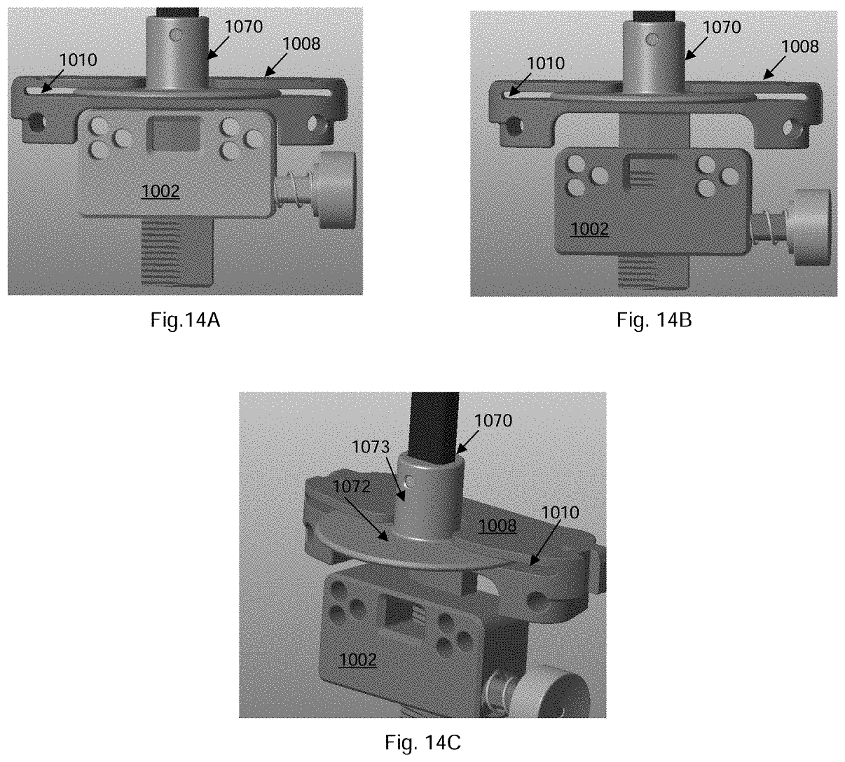

Head 1008 is mounted to body 1002 via a resection level translation mechanism comprising an extension member 1012 coupled to the head 1008. Extension member 1012 mounts in and slides along a channel (see FIGS. 12 and 13 and channel 1009) formed in body 1002 that extends longitudinally in body 1002. Extension member 1012 may extend through body 1008 (e.g. at 1012A) past the body 1002 opposite extension head 1008. The position of extension member 1012 may be selectively extended or retracted and may be selectively set using a set pin 1014 of the resection level translation mechanism (See FIG. 13 showing a cross section and more detail). Extension member 1012 may be formed with teeth 1013 (see FIGS. 11A, 11B and 13) to engage pin end 1014B. The teeth 1013 may be spaced such as 1 mm apart for setting the resection level. When the body 1002 is mounted to a femur, head 1008 may be extended or retracted to set a resection depth up or down relative to the femur and locked in place. FIGS. 14A and 14B show the body 1002 and extension head 1008 in two different relative positions.

Pin 1014, having button 1014A, is biased (e.g. spring biased with spring 1015) in a normally closed position interacting with extension member 1012 to lock extension member 1012 in a selected position. Pushing on pin 1014 (e.g. via button 1014A) releases extension member 1012 for sliding movement in the channel 1009. Though not shown, a cooperating surface of pin 1014 at or near the end 1014B may be shaped to engage the teeth 1013 (e.g. a v-grooved or other shaped surface) of extension member 1012 to lock it in the selected position (e.g. of FIG. 14B).

In the present example, extendible head 1008 provides top end surface 1016. Top end surface 1016 has an edge 1018 that may be formed with a central notch 1018A to receive a tracking element 1070 within slot 1010 so that the cutting guide 1000 provides a tracking element interface. Central here means longitudinally central relative to body 1002, between top end surface 1016 and an opposite end 1020. The notch 1018A, as a component of the tracking element interface, facilitate a deeper seating of the tracking element in the slot 1010 as shown in FIGS. 14A, 14B and 14C.

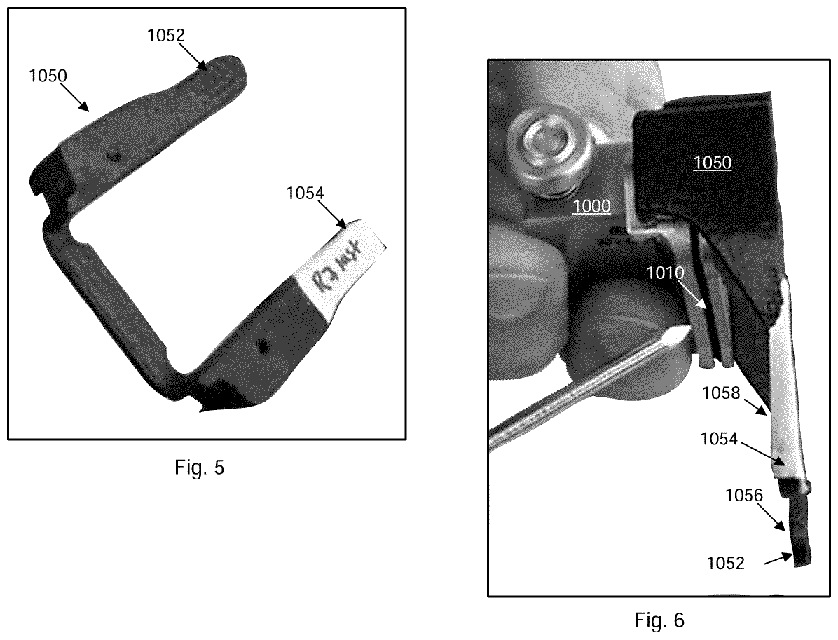

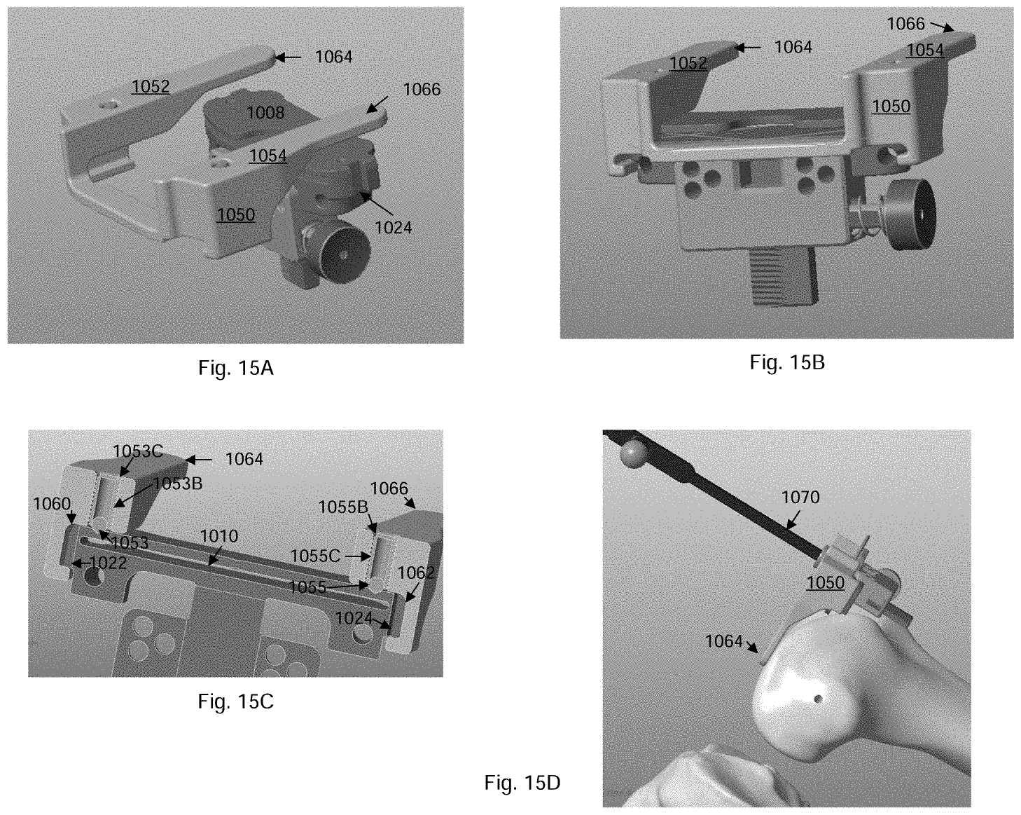

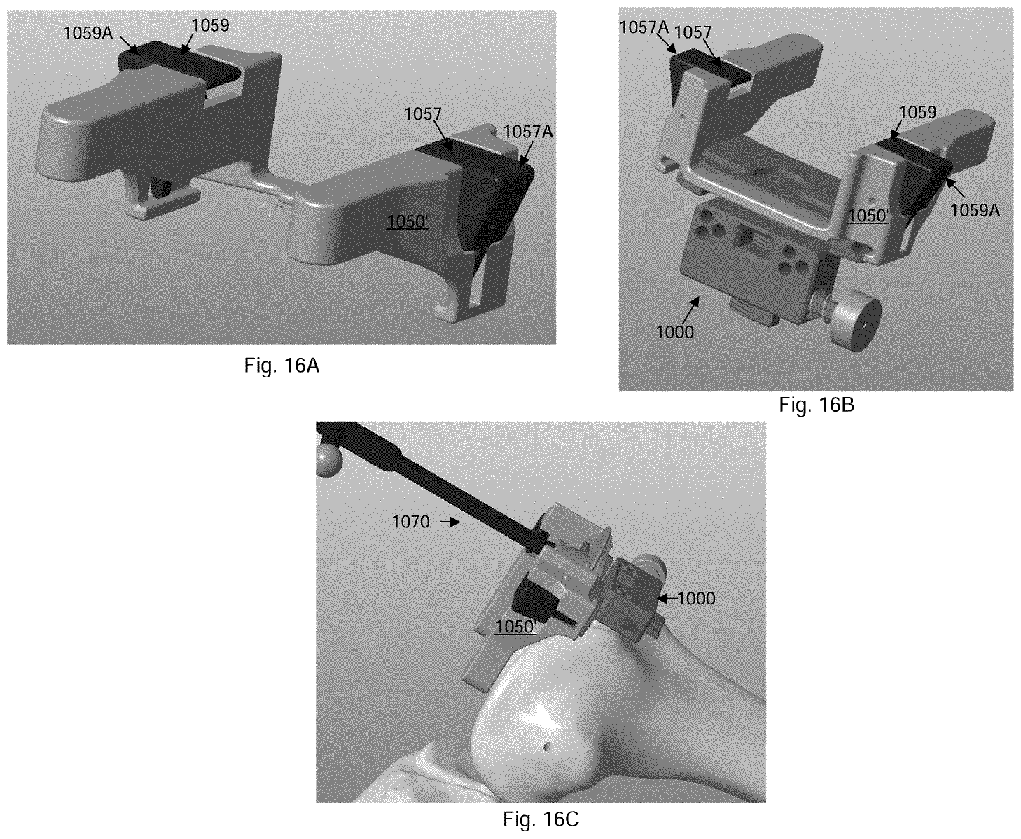

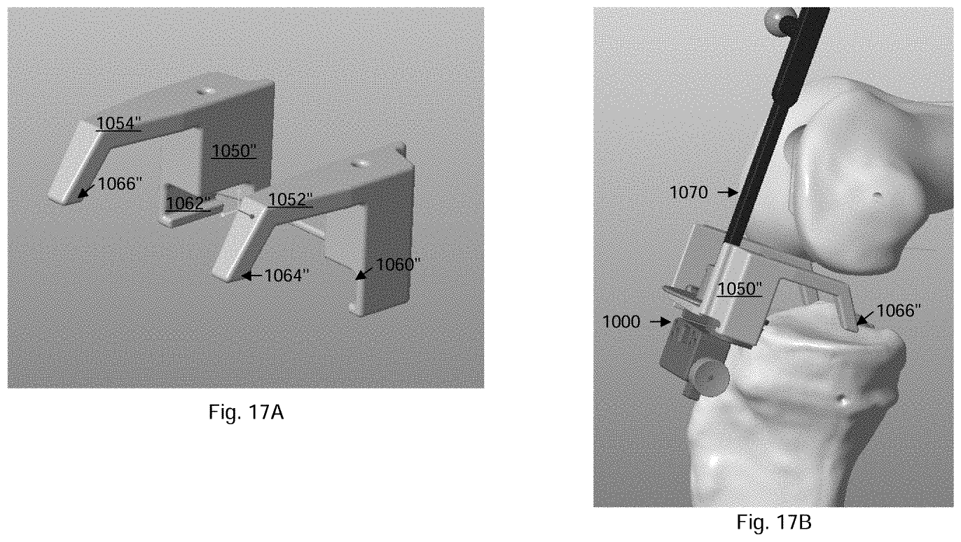

With further reference to FIGS. 5, 6, 11A, 11B, 15A, 15B, 15C, and 15D, cutting device 1000 has a mechanical interface 1022, 1024 to receive a paddle guide 1050 (FIG. 5). It is noted that FIGS. 15A to 15D show one embodiment of paddle guide 1050 while FIGS. 16A to 16C show second embodiment 1050′ and FIGS. 17A to Guide 15B show a third embodiment 1050″. In the drawings and description herein, similar features of the paddle guides are similarly numbered and distinguished using zero, one or two′ symbols.

FIGS. 5, 6 (and others) may show a label (in the form of a piece of tape with text) on paddle guide 1050 which will be understood to be for convenience and not part of the guide's features per se. Mechanical interface 1022 and 1024 is positioned on opposite sides/edges of cutting guide 1000, for example on extendible head 1008. Each mechanical interface 1022 and 1024 comprises a protrusion to engage a cooperating channel on paddle guides 1050, 1050′ and 1050″. See channels 1060 and 1062 of paddle guide 1050 in FIG. 15C.

Each of paddle guides 1050, 1050′ and 1050″ is generally U-shaped to fit around the cutting guide 1000 (e.g. around extendible head 1008) arms of the U-shape provide spaced, opposing and generally parallel extending paddles (e.g. 1052 and 1054, 1052′ and 1054′, and 1052″ and 1054″, respectively) which extend beyond and way from cutting device 1000, generally orthogonally from the longitudinal axis of device 1002 when paddle guide 1050, 1050′ 1050″ is mounted. It is apparent that arms 1052″ and 1054″ crook, like a bent finger, to curl ends 1064″ and 1066″ toward a bone when the guide 1000 is assembled and mounted with the paddle guide 1050″.

A paddle guide may be configured to fit head 1008 in a variety of manners. In one manner paddle guide (e.g. 1050) may be plastic or other flexible material to friction fit head 1008. In one manner (as seen in FIG. 15C showing a cross-section of paddle guide 1050 and head 1008), a friction fit is provided via ball detents 1053 and 1055 having positioning means 1053A and 1055A in channels 10536 and 10556, for example. In one manner, there may be provided a mechanical spring type (biasing) mechanism 1057, 1059, where paddled guide is configured to slide on head 1008, and a button is activated (pressed) to release the mechanism to permit removal.

The tracking element interface (e.g. slot 1010 and notch 1018A), the mechanical interface 1022 and 1024, and the respective tracking element 1070 and paddle guide 1050 are respectively configured to enable assembly and disassembly of the tracking element and the paddle device with the cutting guide device in any order. The U-shape of the paddle guide may engage the mechanical interface and wrap around the cutting guide device without blocking the tracking element interface which receives the tracking device.

When paddle guide 1050 is mounted, as seen mounted in FIG. 6, respective lower surfaces 1056 and 1058 of paddles 1052 and 1054 are parallel to and spaced 9 mm from slot 1010, a clinically relevant number for a default or standard resection depth. In an alternative configuration, the paddles 1052 and 1054 may not extend in a plane parallel to a plane of slot 1010 and may be shaped or canted to fit a distal femur shape (e.g. about exposed condyles or cooperating portions of a tibial plateau), etc. See e.g. FIG. 17B.

Surfaces 1056 and 1058 of paddles 1052 and 1054 may contact the bone (e.g. femur) along a planar surface, and the predefined relative position of the slot is non-coplanar in the varus/valgus direction to mimic the difference between the joint line and mechanical axis of a patient's knee.

Though describe primarily in relation to a distal femur, the cutting guide and paddle guide may be used with a procedure for a proximal tibia. Thus, to facilitate a procedure on the distal femur, the paddles may be configured to contact respective distal condyles (the medial and lateral condyles) of a knee. To facilitate a procedure on the proximal tibia, the paddles may be configured to contact medial and lateral tibial compartments. In an alternative tibia-specific design: instead of flat paddles, the paddles may be configured as one or two curved finger(s) that respectively come down to touch a respective point on the tibial plateau.

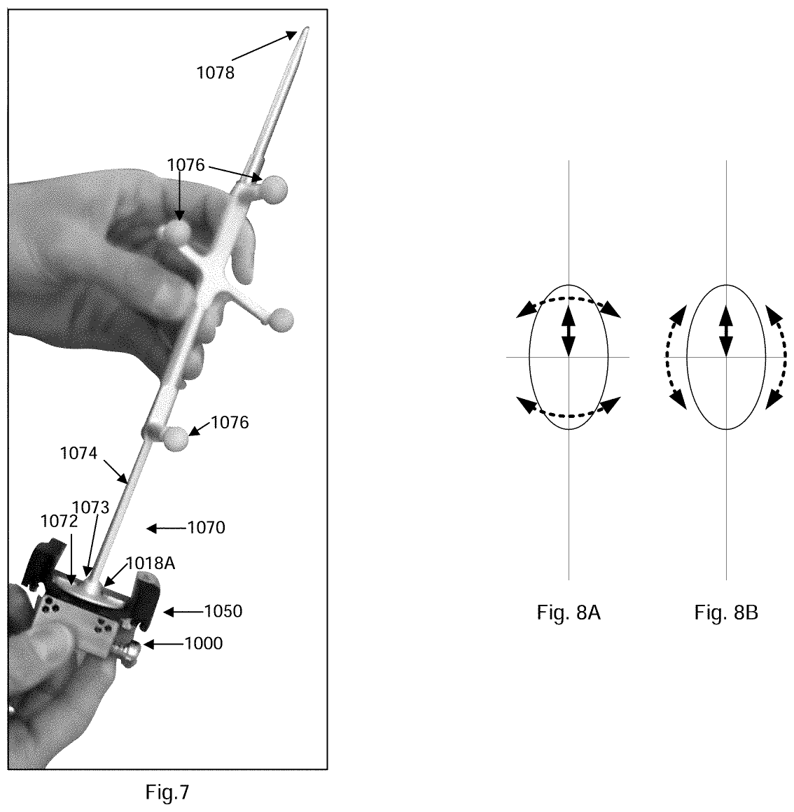

FIGS. 7 (also 15D), 16C and 17B shows a tracking element 1070 mounted to cutting guide 1000 with mounted paddle guide 1050, 1050′ and 1050″ respectively. Tracking element 1070 comprises a flat circular plate or base 1072 to slide in slot 1010 and engage notch 1018A at a central collar 1073 of base 1072. Collar 1073 mounts a tracking element stem 1074, which extends orthogonally from flat base 1072 and carries spaced reflective sensors 1076. A distal end 1078 may be shaped to form a probe tip. See too FIG. 14C for a close-up view of base 1072, collar 1073 and stem 1074.

The spacing (configuration) of tracking element 1070 is known to a surgical navigation system (e.g. provided to and/or stored) such that a pose of the sensors 1076 indicates a pose of base 1072 and particularly the planar bottom surface of base 1072 (not shown). In turn, when mounted to slot 1010, the pose of the slot is known.

With the configuration and relationship between the cutting guide 1000 and paddle guide 1050 when mounted, the paddles 1052 and 1054 may be positioned on respective exposed distal condyles of a femur to guide an initial position of the cutting guide. Measurements are taken to determine flexion/extension first, varus/valgus, and resection depth. The positioning of cutting guide may be guided with audio output as described earlier. FIG. 8A is a lateral schematic view of a knee where the dotted lines show flexion and extension movement and the solid line shows resection depth along a femur. FIG. 8B is a frontal schematic view of a knee where the dotted lines show varus/valgus angle and the solid line shows resection depth along a femur.

In this present example to mount cutting guide 1000 to a femur, an order of steps may be guided using selectively removable paddle guide 1050 as well as workflow (e.g. as provided through a user interface of a surgical navigation system to sequentially direct mounting through target parameters for flexion/extension angle first, then varus/valgus angle, and lastly resection depth. It is understood that other examples may be independent of the order of steps.

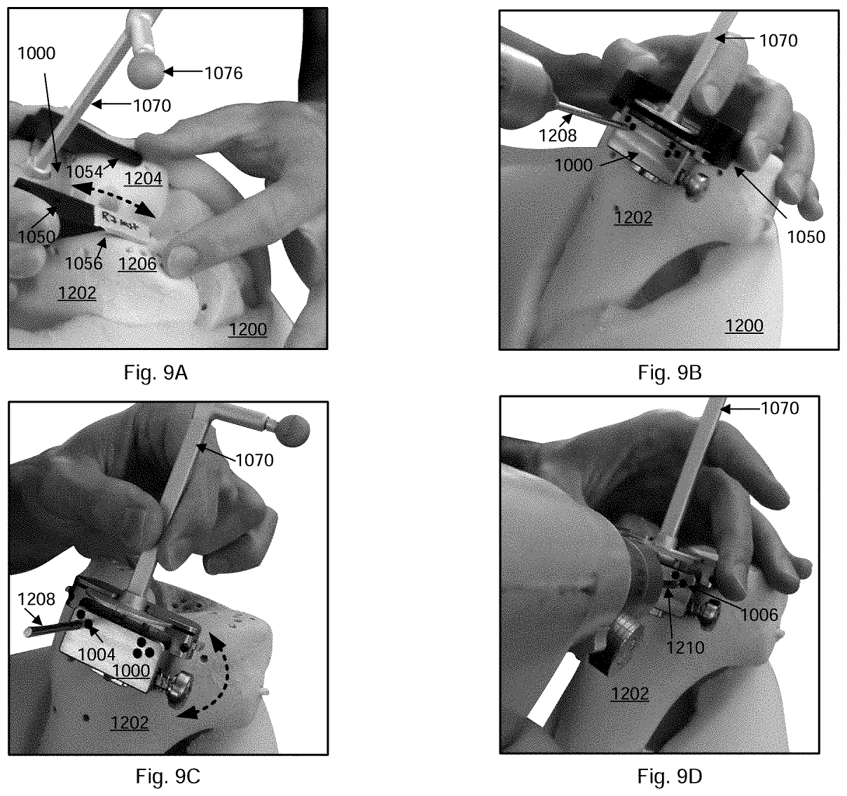

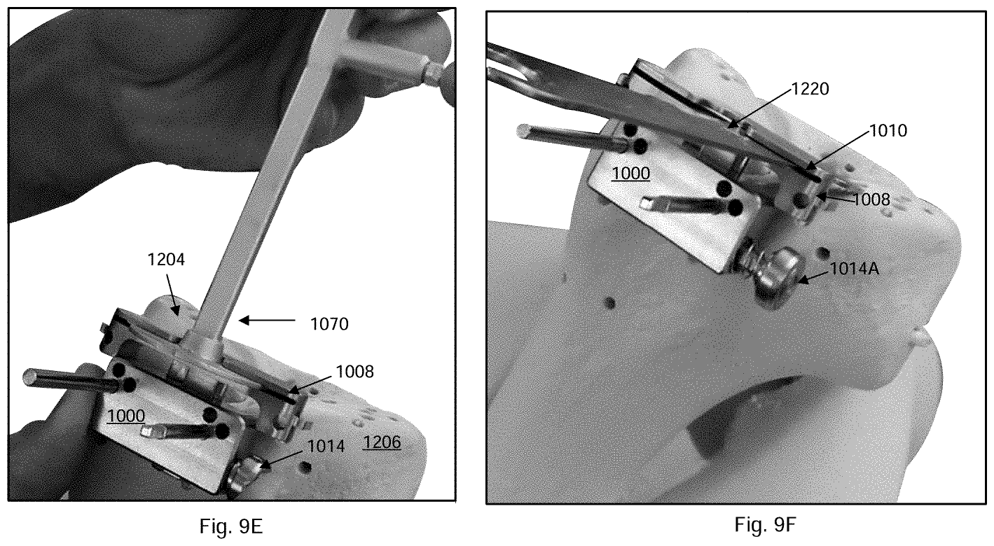

FIGS. 9A to 9F show a model receiving a procedure using the cutting guide, paddle guide and tracking element shown in FIG. 7 according to an embodiment. FIGS. 9A shows a model knee 1200 exposed to show a femur 1202 and ready to receive a cutting guide 1000 as well as showing cutting guide with paddle guide 1050 mounted thereto. Surfaces 1054 and 1056 are engaging the respective condyles (lateral and medial) 1204 and 1206 and may be moved therealong as indicated by dotted line to set the flexion extension. This movement is tracked by surgical navigation system having registered a reference element and received data for the tracking element and target parameters etc. The surgical navigation system, e.g. through programmed workflow, may provide a user interface such as a graphical user interface (GUI) with or without audio output to guide the positioning of cutting guide 1000. The GUI may be provided to a display device. The surgeon may view the GUI or listen for the sounds as the cutting guide is iteratively positioned to meet the target flexion/extension angle parameter.

Once set, the cutting guide is held and a pin 1208 driven in one of the pin holes (e.g. one of pin holes 1004 as shown or pin holes 1006) to fix the flexion/extension angle as shown in FIG. 9B.

Next, FIG. 9C shows paddle guide 1050 removed but tracking element 1070 remaining. This permits cutting guide 1000 to rotate on pin 1208 as shown, by a dotted line, to position varus/valgus angle.

Through navigational guidance (e.g. workflow, GUI with or without sound as described) the target varus/valgus angle parameter may be met. The cutting guide 1000 is held and, as shown in FIG. 9D, a second pin 1210 is driven to fix the position of body 1002 of cutting guide 1000 to femur 1202.

Resection depth may be set, through navigational guidance, so that the resection depth parameter is met. Fig. Guide 7E shows an adjustment to depth to move head 1008 while squeezing set pin 1014. Tracking element 1070 may be used to pull or push on head 1008.

Lastly, the tracking element 1070 is removed and a saw blade 1220 inserted to cut the femur (e.g. epicondyles) as shown in FIG. 9F (enlarged for clarity).

The cutting guide and paddle guide are configured to avoid invasion of the intra-medullary (IM) canal occasioned with traditional IM knee guides. Traditional IM knee guides violate the IM canal with a rod (drilling and placed in the IM canal) to act as a reference axis to position the cutting slot of the IM guide before pinning such as to an anterior femur.

In contrast the cutting guide and method as disclosed herein uses navigation and registration, such that there is no need to violate the canal. Violating the canal has downsides, including:
more blood loss;
more invasive;
no control of flexion/extension of cutting guide;
any varus-valgus adjustment is in reference to femur canal axis as determined by the implanted IM reference rod, which may vary from patient to patient from the “gold standard” mechanical axis.

Hence, when mounting cutting guide 1000 using navigation, only two pins need to be used and the resection level is adjustable after the cutting guide 1000 is mounted via the pins.

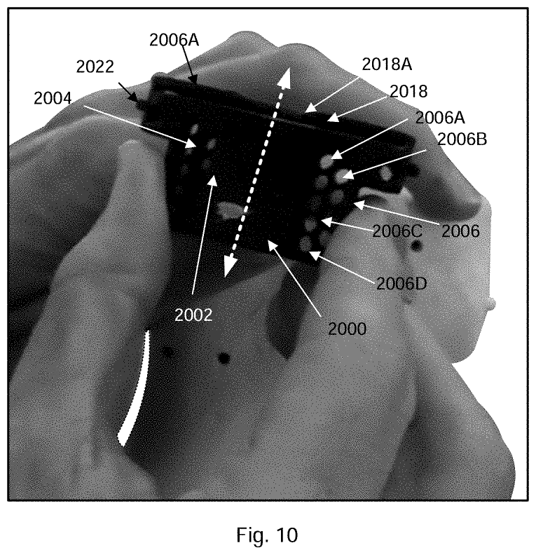

An alternatively configured and simplified cutting guide does not have a translation mechanism to set resection depth by moving a head relative to a body. FIG. 10 shows cutting guide 2000 having a body 2002 with a plurality of spaced pin holes 2004 and 2006 similar to pin holes 1004 and 1006 on opposite sides (spaced relative to the longitudinal axis represented by the dotted line) of body 2002. No moveable head is shown. A slot 2010 is formed in one end of body 2002 near surface 2018 having notch 2018A, similar to cutting device 1000. Also present is mechanical interface 2022, similar to mechanism 1022 to mount a paddle guide.

Within each plurality of spaced pin holes 2004 and 2006, the respective pin holes (e.g. 2006A, 2006B, 2006D, etc. are also spaced longitudinally along body for example in two columns slightly offset to allow fine movement of the device 2000. In this way, cutting guide 2000 may be mounted, under navigational guidance, to set flexion/extension angle and varus/valgus angle as previously described. To set resection depth, the cutting guide may be removed (e.g. by sliding over the mounting pins) and appropriate pin holes from spaced pin holes 2004 and 2006 selected that provide the depth to meet the target resection depth parameter. In a respective cluster of pin holes, a first pin hole may be spaced an initial distance X mm from the top surface and/or slot, a second pin hole spaced (X+Y) mm and a third pin hole spaced (X+2Y) mm from the top surface, etc. to give measured adjustment. The Y value may be 1 for example, to allow 1 mm adjustment.

Thus there is provided a cutting guide device for TKA comprising: opposite ends and a longitudinal axis therebetween; a planar blade guide surface to receive a blade of an oscillating saw, the planar blade guide surface formed adjacent a first end of the opposite ends, the planar blade guide surface extending transversely to the longitudinal axis; a mechanical interface to couple a paddle device, the paddle device configured to contact one of a distal femur and a proximal tibia to locate the cutting guide device adjacent the one of the distal femur and proximal tibia to mount the cutting guide device thereto; at least two pin holes formed through the cutting guide device to receive respective pins to mount the cutting guide device; a resection depth translation mechanism enabling translation of the planar blade guide surface along a direction of the longitudinal axis; and a tracking element interface to receive a tracking element, the tracking element interface having a predefined positional relationship with the planar blade guide surface.

The paddle device may comprise a pair of spaced paddles extending from the paddle device for contacting the one of the distal femur and the proximal tibia; and the mechanical interface and paddle device may be respectively configured such that when the cutting guide device is assembled with the paddle device via the mechanical interface, the planar blade guide surface is in a predefined position relative to the pair of spaced paddles. In relation to the planar blade guide surface, the predefined position relative to the pair of spaced paddles may be based on a default resection depth of a knee implant. In one example, the pair of spaced paddles may contact the one of the distal femur and the proximal tibia along a planar surface thereof, and the predefined position may be co-planar. In one example, the pair of spaced paddles may contact the one of the distal femur and the proximal tibia along a planar surface, and the predefined relative position of the planar blade guide surface is non-coplanar in a varus/valgus direction to mimic a difference between a joint line and a mechanical axis of a patient's knee.

To facilitate a procedure on the distal femur, the pair of spaced paddles may be configured to contact medial and lateral condyles of a knee; and to facilitate a procedure on the proximal tibia, the paddles may be configured to contact medial and lateral tibial compartments.

The tracking element interface, the mechanical interface and, respectively, the tracking element and the paddle device may be configured to enable assembly and disassembly of the tracking element and the paddle device with the cutting guide device in any order. The paddle device may be U-shaped to engage the mechanical interface and wrap around the cutting guide device without blocking the tracking element interface to receive the tracking element.

The tracking element interface may comprise the planar blade guide surface and the tracking element may provide a mating surface for planar contact with the planar blade guide surface.

The planar blade guide surface may provide a surface of a planar slot formed in the cutting guide device transversely to the longitudinal axis. The planar slot may be adjacent the first end and the first end may define (eg. form) a notch portion in the planar slot as a component of the tacking element interface to facilitate a deeper seating of the tracking element in the planar slot.

The cutting guide device may comprise a first cluster of pin holes and a second cluster of pin holes located opposite and parallel thereto across the longitudinal axis, each of the first cluster and the second cluster spaced in a known parallel spacing along the longitudinal axis and perpendicular to a plane of the planar blade guide surface. The resection depth translation mechanism may comprise the first cluster of pin holes and the second cluster of pin holes.

The resection depth translation mechanism may enable translation of the planar blade guide surface relative to the at least two pin holes along the direction of the longitudinal axis.

The cutting guide device may comprise a body and a head coupled to the body via an extension member, the head carrying the planar blade guide surface and wherein the resection depth translation mechanism may comprise the extension member mounted for sliding in a channel of the body and a locking pin to releasably lock a position of the head relative to the body. The head may further comprise the mechanical interface.

The pin holes may be parallel to each other and the planar blade guide surface.

There is provided a kit comprising a cutting guide device in accordance with any of the examples and embodiments, etc. and at least one of a paddle device and a tracking element. The kit may comprise a plurality of pins.

There is provide a computer implemented method to provide guidance for a TKA procedure on a patient the procedure using a cutting guide device in accordance with any one of the examples and embodiments herein. The method comprises: receiving target parameters associated with a target position for the cutting guide device relative to the one of the distal femur and the proximal tibia; receiving first tracking data responsive to the tracking element where the tracking element, the cutting guide device and the paddle device are in an assembly with the paddle device assembled via the mechanical interface and the tracking element assembled via the tracking element interface; determining measurements relative to the target parameters; and providing navigational guidance responsive to the measurements for positioning the cutting guide device into the target position.

The method may comprise, in response to movement of the tracking element, receiving further tracking data, updating the measurements and providing updated navigational guidance responsive to the measurements as updated.

Providing navigational guidance may comprise providing a user interface comprising audio output to a speaker device to guide positioning the cutting guide device into the target position. Providing navigational guidance may comprises providing a graphical user interface comprising display information to a display device to guide the positioning.

The target parameters may comprise a set of target step parameters defining an ordered set of target step positions to position the cutting guide device in the target position; and the method may comprise using workflow to provide navigational guidance to position the cutting guide device assembled with the tracking element in each of the target step positions and in an order responsive to the ordered set. The method may comprise: receiving input indicating a completion of positioning of the cutting guide device in one of the target step positions and readiness for navigational guidance to position the cutting guide device in a next target step position associated with a next one of the set of target step parameters in accordance with the workflow; and, responsive to the input, providing navigational guidance for the next target step position in response to measurements determined relative to the next one of the set of target step parameters.

The ordered set of target parameters may comprise: flexion/extension angle, varus/valgus angle and resection depth.

There is provided a computing device configured to perform the method to provide guidance for a TKA procedure on a patient the procedure using a cutting guide device in accordance with any one of the examples and embodiments herein. There is provided a computer program product comprising a non-transient storage unit (e.g. a memory, disk/disc, etc.) storing instructions which when executed by a processing unit of a computing device configure the computing device to perform the method.

There is provided a method of using the cutting guide device according to the examples and embodiments herein. The method comprises: holding an assembly of the cutting guide device, the paddle device and the tracking element where the paddle device is assembled via the mechanical interface and the tracking element is assembled via the tracking interface; positioning the assembly so that the paddle device contacts a bone defined by the one of the distal femur and the proximal tibia and with the two pin holes aimed at the bone in a first position thereon; adjusting the assembly into a first target position relative to the bone based on navigational guidance; partially securing the cutting guide device to the bone by driving a bone pin into the bone through a first pin hole; removing the paddle device from the assembly; adjusting the assembly into a second target position relative to the bone based on navigational guidance; securing the cutting guide device to the bone in the second target position by driving a bone pin into the bone through an opposite pin hole spaced and parallel to the first pin hole; while maintain the assembly in the second target position, adjusting the assembly into a third desired position relative to the bone based on navigational guidance using the resection depth translation mechanism; positioning a saw blade along the planar blade guide surface; and cutting the bone. The first target position may be associated with a flexion/extension angle target parameter, the second target position may be associated with a varus/valgus angle and the third target position is associated with a resection depth target parameter.

Various alternative or additional approaches may be contemplated and combined with the two primary examples described herein.

As noted, various audio signals could be used for feedback relative to movement of the tracked device and the target parameters. The audio signal may comprise beeping, may be directional to suggest a corrective direction to achieve the target parameter. Different states may be indicated with different sounds. For example, to indicate the device is positioned out of range (e.g. beyond a threshold about the target parameter), in range or on-target (e.g. within the threshold of the target parameter) or not tracking (e.g. because one or more sensors is not visible in an image, etc.)

The user interface may be interactive and receive input (e.g. from a user), for example, to toggle between which measured parameters are “active” in terms of generating audio signals. The interface may toggle or otherwise switch/select which target parameter is active.

Audio signals may be “composite”, representing more than one measured parameter (e.g. high pitch beeping for one parameter, low pitch beeping for another). There may be configured a single audio signal representing more than one measured parameter (e.g. the audio signal could be determined based on the norm of the flexion extension/varus valgus angles).

The GUI may graphically indicate the target parameters and measured parameters as the device is tracked during positioning. The GUI may be configured to indicate graphically which measured parameter is “actively” generating sounds. A graphic element may pulse, whether alone or in synchronicity with an audio signal to guide placement. The GUI may be configured to receive input to disable audio output when guiding placement.

In addition to computing device aspects, a person of ordinary skill will understand that computer program product aspects are disclosed, where instructions are stored in a non-transient storage device (e.g. a memory, CD-ROM, DVD-ROM, disc, etc.) to configure a computing device to perform any of the method aspects stored herein.

Practical implementation may include any or all of the features described herein. These and other aspects, features and various combinations may be expressed as methods, apparatus, systems, means for performing functions, program products, and in other ways, combining the features described herein. A number of embodiments or examples have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments or examples are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers characteristics, compounds, chemical moieties or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

1-33. (canceled)

34. A computer method for a guided procedure on a patient comprising:
receiving at least one target parameter;
determining at least one measured parameter in real time using data received from a navigation system;
generating an audio signal indicative of a proximity of the measured parameter to the target parameter; and
providing the audio signal to a speaker device.

35. The method of claim 34 wherein the audio signal indicates the proximity of the measured parameter with the target parameter in accordance with one of:
a beeping tone wherein a frequency is one of proportional and inversely proportional to a magnitude of difference between the measured parameter and the target parameter;
a different signal indicating if the difference is positive or negative where the difference is positive when the measured parameter is greater than the target parameter and negative when less than the target parameter;
a distinct audio signal when the target parameter=measured parameter, within a threshold thereof;
a distinct audio signal when the measured parameter is grossly out of range of the target parameter such that the difference is >>an out of range threshold; and
a distinct audio signal indicating a “Not Tracking” state of the navigation system, whereby the navigation system is unable to produce the data to determine the measured parameter.

36. The method of claim 34 wherein there are two or more target parameters associated with two or more measured parameters, and the audio signal is generated based only in respect of one of the two or more target parameters associated with two or more measured parameters.

37. The method of claim 36 wherein the method includes receiving user input to switch between one of the two or more target parameters and another one of the two or more target parameters.

38. The method of claim 34 wherein the method includes providing a user interface to receive input to configure the audio signal.

39. The method of claim 38 wherein an option to configure the audio signal comprises an option to turn on/off audio output and/or adjust volume.

40. The method of claim 34 wherein both the target parameter and the measured parameter are provided for display to a display screen, wherein the display comprises one or more of graphical or numerical representation.

41. The method of claim 40 wherein one of the two or more target parameters is displayed and graphically emphasized.

42. The method of claim 41 wherein a plurality of the two or more measured parameters is displayed, and the one of the two or more target parameters is emphasized by:
an increased brightness on the display screen;
a coloured or thicker border; and
a graphical element associated with of the two or more target parameters that changes its visual appearance synchronously with the audio signal (e.g. a flashing border that flashes with each beep).

43. The method of claim 34 wherein the guided procedure is a Total Knee Replacement, and the at least one target and at least one measured parameter are chosen from:
varus/valgus angle;
flexion/extension angle;
resection depth; and
overall leg alignment.

44. The method of claim 34 wherein the guided procedure is a Total Hip Arthroplasty, and the at least one target parameter and at least one measured parameter are chosen from:
leg length;
offset;
acetabular inclination;
acetabular anteversion;
acetabular reaming depth; and
broach depth.

45. The method of claim 34 wherein the procedure comprises a pedicle screw placement, and the at least one target parameters are useful to align a trajectory of a tool with a pedicle of the patient.

46. A computing device comprising a processing unit coupled to a storage device storing instructions, which, when executed by the processing unit, configure the computing device to:
receive at least one target parameter;
determine at least one measured parameter in real time using data received from a navigation system;
generate an audio signal indicative of a proximity of the measured parameter to the target parameter; and
provide the audio signal to a speaker device.

47. A computer program product comprising a non-transient storage unit storing instructions, which, when executed by a processing unit of a computing device, configure the computing device to:
receive at least one target parameter;
determine at least one measured parameter in real time using data received from a navigation system;
generate an audio signal indicative of a proximity of the measured parameter to the target parameter; and
provide the audio signal to a speaker device.
